(19) United States
(12) Patent Application Publication
(10) Pub. No.: US 2021/0000815 A1
(43) Pub. Date: Jan. 7, 2021

(54) PHARMACOKINETIC ENHANCEMENT OF EZH2 INHIBITORS THROUGH COMBINATION THERAPIES

(71) Applicant: Constellation Pharmaceuticals, Inc., Cambridge, MA (US)

(72) Inventor: Adrian Senderowicz, Somerville, MA (US)

(21) Appl. No.: 16/958,468

(22) PCT Filed: Dec. 27, 2018
(86) PCT No.: PCT/US2018/067623

§ 371 Date: Jun. 26, 2020

Related U.S. Application Data
(60) Provisional application No. 62/611,119, filed on Dec. 28, 2017.

(51) Int. Cl. A61K 31/4545; A61K 31/5377

(52) U.S. Cl. CPC A61K 31/4545; A61K 31/5377

(57) ABSTRACT

Provided herein are pharmaceutical composition comprising an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof. Also provided s the use of an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof for treating cancer.

### RELATED APPLICATIONS

This application claims the benefit of priority to U.S. Provisional Application No. 62/611,119, filed Dec. 28, 2017, the entire contents of which are incorporated herein by reference.

### BACKGROUND

(R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, referenced herein as Compound 1, is an effective inhibitor of Enhancer of Zeste Homolog 2 (EZH2) and is useful in treating a variety of solid tumors and hematologic malignancies. See e.g., U.S. Pat. No. 9,085,583, the contents of which are incorporated herein by reference. A drawback to Compound 1, however, is that it has a relatively short half-life and reduced exposure upon multiple-dose administration (approximately 60% reduction in AUC with multiple doses). This has led to the unpleasant burden to administer multiple dosages of Compound 1 at one time, the frequent administration of such dosages such as two or three times a day, and chronic substandard concentrations that could prevent long term activity. In an effort to relieve this burden and facilitate strict patient compliance, and to improve chances for long term antitumor activity, the need exists for improved drug exposure.

### SUMMARY

It has now been found that Compound 1 is primarily metabolized by the hepatic enzyme CYP3A, and that Compound 1 also appears to be a stronger inducer of CYP3A. Co-administration of Cobicistat (Tybost®) has now been found to enhance the pharmacokinetics of Compound 1. As such, effective amounts of cobicistat with effective amounts of Compound 1 can now be used to improve the exposure of Compound 1 and alleviate the current need to administer multiple dosages as one time. Provided herein, therefore, are compositions comprising effective amounts of cobicistat and effective amounts of Compound 1, and their use for the treatment of one or more cancers.

The predominant effects resulting from the administration of cobicistat which led to an increase in the exposure of Compound 1 were an increase in the minimum blood plasma concentration (Cₘᵢₙ), an increase in the maximum serum concentration (Cₘₐₓ), an increase in the actual body exposure represented by the area under the curve (AUC), and a reduction in the apparent volume of distribution (V_{z}/F). For example, in one aspect, the Cₘᵢₙ, Cₘₐₓ, and AUC of Compound 1 increased by an average of over 29-fold, over 3-fold, and over 6-fold respectively when used in combination with cobicistat. See e.g., FIG. 3. In addition, the V_{z}/F of Compound 1 decreased by an average 6-fold. See e.g., FIG. 3. These results have important implications because they influence the dosing frequency of the boosted drug and the dose of cobicistat needed for adequate pharmacokinetic enhancement.

Interestingly, however, the use of Compound 1 with cobicistat has some markedly different effects when compared to co-administration with antivirals such as elvitegravir and darunavir. For example, while cobicistat increases the half-lives (T_{1/2}) of these drugs by 3- to 4-fold, it had little or no effect on the T₁₁₂ of Compound 1. Moreover, the volume of distribution resulting from the combination of Compound 1 and cobicistat is significantly different than with the antivirals (a six fold reduction versus a maximal three fold reduction for the antivirals). See e.g., FIG. 3.

### BRIEF DESCRIPTION OF THE FIGURES

FIG. 1 is a table summarizing the results of the single-dose Compound 1 pharmacokinetic parameters with and without cobicistat pretreatment in healthy volunteers. CBST=cobicistat pretreatment and Compound 1 was administered as single doses of 400 mg; first alone and then following three daily doses of cobicistat 150 mg with a 7-day washout between Compound 1 doses.

FIG. 2 is a table summarizing the comparison of mean single-dose pharmacokinetic parameters for Compound 1 when Compound 1 was administered as a 1600 mg dose to lymphoma patients in study 01 and as a 400 mg dose with cobicistat pretreatment to healthy volunteers in study 02.^{a}=Compound 1 400 mg was administered after 3 once daily doses of cobicistat.

FIG. 3 is a table summarizing the comparative pharmacokinetic boosting effects of ritonavir on various antivirals versus the pharmacokinetic boosting effects of cobicistat on Compound 1.

FIG. 4 is a table summarizing the mean pharmacokinetic parameters for Compound 1 in combination with other agents and with and without cobicistat in human subjects.

### DETAILED DESCRIPTION

Provided herein are pharmaceutical compositions comprising an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof.

Also provided are pharmaceutical compositions comprising an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof; an effective amount of cobicistat, or a pharmaceutically acceptable salt thereof; and a pharmaceutically acceptable carrier.

Also provided herein is method for treating cancer in a subject, comprising administering to the subject an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof.

Also provided herein is the use of an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof, in the manufacture of a medicament for treating cancer.

Also provided is an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof, for use in treating cancer.

Further provided is a pharmaceutical composition comprising an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof, for treating cancer.

(R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, referred to herein as Compound 1, has the following chemical structure:

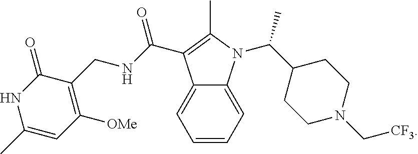 [SMILES: COc1cc(C)[nH]c(=O)c1CNC(=O)c1c(C)n([C@H](C)C2CCN(CC(F)(F)F)CC2)c2ccccc12]

The term “pharmaceutically acceptable carrier” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Pharmaceutically acceptable salt forms of Compound 1 and/or cobicistat include pharmaceutically acceptable acidic/anionic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids).

Unless otherwise indicated, in the present methods and uses, an effective amount of Compound 1 may be administered prior to, concurrently with, or after administration of cobicistat. Thus, simultaneous administration is not necessary for therapeutic purposes. In one aspect, however, Compound 1 is administered concurrently with cobicistat. In another aspect, the subject is pre-treated with cobicistat (i.e., Compound 1 is administered after cobicistat) for a period of time such as e.g., 0.5, 1.0, 1.5, 2.0, 2.5, 3.0, 3.5, 4.0, 4.5, 5.0, 5.5, or 6.0 hours or greater before Compound 1 is administered to the subject.

As used herein, the terms “treatment,” “treat,” and “treating” refer to reversing, alleviating, or inhibiting the progress of a cancer, or one or more symptoms thereof.

Exemplary types of cancer include e.g., adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In one aspect, the cancer to be treated is selected from melanoma, prostate cancer, breast cancer, colon cancer, ovarian cancer, bladder cancer, lung adenocarcinoma, and carcinoma of the pancreas. In another aspect, the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL).

In one aspect, the cancer to be treated is a lymphoma, leukemia, or a solid tumor such as in prostate cancer.

In one aspect, the cancer to be treated is selected from Hodgkin's lymphoma, non-Hodgkin's lymphoma, chronic lymphocytic leukemia, and multiple myeloma.

In one aspect, the cancer to be treated is a lymphoma or a solid tumor such as in prostate cancer.

In another aspect, the cancer to be treated is non-Hodgkin's lymphoma.

The term “effective amount” or “therapeutically effective amount” refers to an amount of a compound described herein that will elicit a biological or medical response of a subject e.g., a dosage of between 0.001-100 mg/kg body weight/day.

The term “patient,” as used herein, means an animal, such as a mammal, and such as a human. The terms “subject” and “patient” may be used interchangeably.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In one aspect, the compositions described herein are formulated for oral administration.

Other forms of administration are as described in WO 2013/075083, WO 2013/075084, WO 2013/078320, WO 2013/120104, WO 2014/124418, WO 2014/151142, and WO 2015/023915, the contents of which are incorporated herein by reference.

In one aspect, the effective amount of Compound 1 and/or cobicistat that is administered to a subject, or the effective amount of Compound 1 and/or cobicistat that is formulated in a provided composition, is such that a dosage of between 0.01-100 mg/kg body weight/day can be administered to the subject. It will be understood that a specific dosage and treatment regimen for any particular patient will depend upon a variety of factors, including age, body weight, general health, sex, diet, time of administration, rate of excretion, drug combination, the judgment of the treating physician, and the severity of the cancer being treated.

In one aspect, the effective amount of Compound 1 ranges from 50 mg to 3.5 grams administered once, twice, or three times a day. For example, in one aspect, the effective amount of Compound 1 ranges from 100 mg to 3.5 grams, from 200 mg to 3.0 grams, from 200 mg to 2.5 grams, from 200 mg to 1.0 grams, from 200 mg to 1.5 grams, from 200 mg to 1 gram, from 300 mg to 800 mg, from 400 mg to 800 mg, or from 400 mg to 600 mg administered once, twice, or three times a day. In another aspect, the effective amount of Compound 1 ranges from 100 mg to 3.5 grams, from 200 mg to 3.0 grams, from 200 mg to 2.5 grams, from 200 mg to 1.0 grams, from 200 mg to 1.5 grams, from 200 mg to 1 gram, from 300 mg to 800 mg, from 400 mg to 800 mg, from 400 mg to 600 mg, from 1.0 grams to 2.0 grams, from 1.4 grams to 1.7 grams, from 500 mg to 700 mg, from 550 mg to 650 mg, from 700 mg to 900 mg, from 750 mg to 850 mg, from 300 mg to 500 mg, or from 350 mg to 450 mg administered once, twice, or three times a day. In another aspect, the effective amount of Compound 1 is 3.5 grams, 3.4 grams, 3.3 grams, 3.2 grams, 3.1 grams, 3.0 grams, 2.9 grams, 2.8 grams, 2.7 grams, 2.6 grams, 2.5 grams, 2.4 grams, 2.3 grams, 2.2 grams, 2.1 grams, 2.0 grams, 1.9 grams, 1.8 grams, 1.7 grams, 1.6 grams, 1.5 grams, 1.4 grams, 1.3 grams, 1.2 grams, 1.1 grams, 1.0 grams, 900 mg, 800 mg, 700 mg, 600 mg, 500 mg, 400 mg, or 300 mg administered once, twice, or three times a day. In another aspect, the effective amount of Compound 1 is 3.2 grams, 2.4 grams, 1.6 grams, 1.2 grams, 800 mg administered once, twice, or three times a day. In another aspect, the effective amount of Compound 1 is 1.6 grams administered twice a day, 800 mg administered three times a day, 800 mg administered twice a day, 600 mg administered twice a day, or 400 mg administered twice a day.

In one aspect, the effective amount of cobicistat ranges from 75 mg to 500 mg administered once, twice, or three times a day. In another aspect, the effective amount of cobicistat ranges from 100 mg to 400 mg, from 100 mg to 300 mg, and from 100 to 200 mg. administered once, twice, or three times a day. In another aspect, the effective amount of cobicistat is 150 mg administered once a day. In another aspect, the effective amount of cobicistat is 150 mg administered twice a day.

In one aspect, 1.6 grams, 800 mg, 600 mg, or 400 mg of Compound 1 is administered to the subject once or twice a day, and 150 mg of cobicistat is administered to the subject once a day.

### Exemplification

(R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide can be made according to the procedure set forth in U.S. Pat. No. 9,085,583, the contents of which are incorporated herein by reference.

### Effects of Cobicistat on Exposure of Compound 1 in Healthy Subjects

Healthy subjects were treated with Compound 1 with or without pretreatment with cobicistat. A single 400 mg dose of Compound 1 was administered under fasted conditions. There was a 7-day washout between administrations of the two single doses of Compound 1. Compound 1 was administered alone on Day 1 of Period 1 and administered in the presence of cobicistat on Day 1 of Period 2. Cobicistat was administered at a dose of 150 mg once daily for three days, with the third dose preceding the single dose of Compound 1 by two hours. Blood samples were collected for pharmacokinetic analysis of Compound 1 on Day 1 of each period at the following time points 0 (predose), 0.5, 1, 1.5, 2, 3, 4, 6, 8, 10, and 24 hours after the single dose of Compound 1.

All six subjects were males between the ages of 31 and 52 years. Three of the 6 subjects (50%) treated with Compound 1 with and without cobicistat reported a total of 6 adverse events. Diarrhea, reported in 2 subjects, was the only adverse event reported by more than one subject. The other adverse events reported by a single subject each included: abdominal pain, back pain, groin pain, and upper respiratory tract infection. Three of the 6 adverse events were considered to be related to study drug: concurrent events of diarrhea and abdominal pain and the other case of diarrhea. All adverse events were reported in Period 2, after dosing of all study drugs had been completed. With the exception of a moderate case of upper respiratory tract infection reported 7 days after receiving the last dose of Compound 1 all adverse events were considered to be mild in severity and resolved without sequelae.

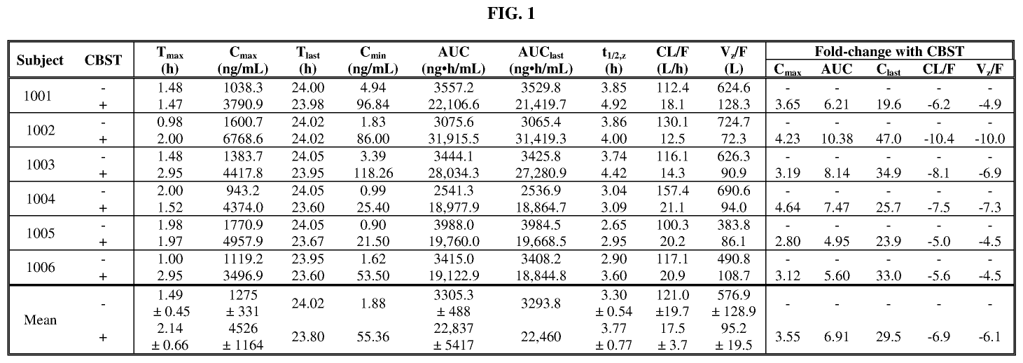

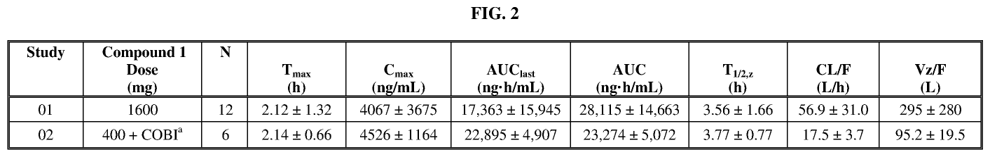

Pre-treatment with cobicistat successfully increased the exposure of 400 mg Compound 1. As shown in FIG. 1, Cₘₐₓ was increased an average of 3.6-fold, while AUC was increased 6.9-fold in the presence of cobicistat. All subjects had at least a 2.8-fold increase in Cₘₐₓ and a 4.9-fold increase in AUC. Cₘᵢₙ was also substantially increased in the presence of cobicistat; all subjects experienced at least a 19-fold increase in Cₘᵢₙ (mean increase of 29-fold). The Tₘₐₓ and terminal half-life were unaffected by the presence of cobicistat. In contrast, clearance and volume of distribution were decreased by 6.9-fold and 6.1-fold, respectively. These results suggest that cobicistat increases Compound 1 exposure via changes in first-pass effect and not as a result of decrease in drug elimination. As shown in FIG. 2, the exposure achieved with a single dose of Compound 1 400 mg with cobicistat 150 mg in healthy subjects was remarkably similar to that achieved with a single 1600 mg dose of Compound 1 alone in patients with lymphoma.

### Comparison of PK Enhancement of Ritonavir/Cobicistat on Antivirals Versus PK Enhancement of Cobicistat on Compound 1

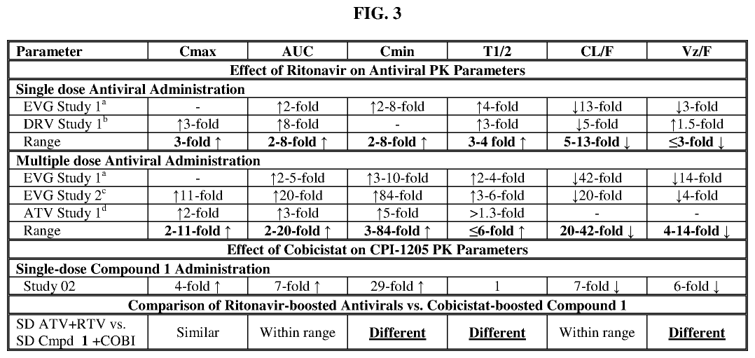

The data in FIG. 3 summarizes the pharmacokinetic boosting achieved with ritonavir (for which cobicistat was found to be bioequivalent) when coadministered with single and multiple doses of elvitegravir (EVG), atazanavir (ATV) and darunavir (DRV), and the pharmacokinetic boosting that COBI achieved when coadministered with single doses of Compound 1.

EVG Study 1 was a multiple ascending dose study of EVG in HIV-infected patients. Six patients per cohort received 50 mg EVG alone or in combination with RTV (100 mg QD) for 10 days. The pharmacokinetics of EVG was assessed on Day 1 (single dose study 1^{a} in FIG. 3) and Day 10 (multiple dose study 1^{a} in FIG. 3). In EVG Study 2, 50 mg EVG was administered alone for the first 10 days and RTV (100 mg BID) was then added for another 10 days in a group of 12 healthy volunteers. The pharmacokinetics of EVG were compared when it was administered alone on Day 10 after a single dose of RTV 100 mg on Day 11 or after 10 days of EVG/RTV 100/100 mg QD on Day 20. Comparative fold effects from this study are shown in FIG. 3.

The pharmacokinetics of 300 mg ATV when administered alone and in combination with RTV 100 mg QD was also evaluated in HIV patients (Achenbach et al., 2011). Comparative fold effects from this study are shown in FIG. 3.

In DRV Study 1, 8 healthy volunteers were treated with a single 150 mg IV dose of DRV with and without 100 mg RTV and a single oral dose of 600 mg DRV without and without 100 mg RTV in a cross-over design. RTV was administered BID for a total of 5 days, starting 2 days prior to DRV administration and ending 3 days after DRV administration. Comparative fold effects from this study are shown in FIG. 3.

As shown from the results, the three pharmacokinetic parameters that were distinct between the use of cobicistat with Compound 1 and the cobicistat equivalent RTV with antivirals was the half-life, minimum blood plasma concentration, and apparent volume of distribution. Interestingly, although cobicistat is a known CYP3A inhibitor, it had little or no effect on the half-life of Compound 1. This is contrary to the data provided for antivirals where the half-lives were extended to the point where their dosing frequency could be reduced to once daily. The half-life of Compound 1 was below 5 hours in all subjects treated with Compound 1 and cobicistat, suggesting that Compound 1 should be dosed at least twice daily.

A substantial difference in the magnitude of increase in trough concentrations of Compound 1 when boosted with cobicistat was also observed in comparison to that achieved with the antivirals and ritonavir. Subjects treated with Compound 1 and cobicistat had a mean change in Cₘᵢₙ of 29-fold compared to a maximum mean increase of 2-fold to 8-fold after single dosing with antivirals and RTV. Finally, the reduction of Compound 1 apparent volume of distribution was also of greater magnitude than the reduction observed with the antivirals (6-fold reduction for Compound 1 versus a maximal 3-fold reduction for the antivirals).

This data shows that cobicistat behaves differently when coadministered with the EZH2 inhibitor Compound 1 than when it is coadministered with antiviral agents, resulting in differences in the terminal half-life, trough concentrations and volume of distribution of Compound 1 compared to that observed with antivirals. Notwithstanding these differences, cobicistat enhances the pharmacokinetics of Compound 1 enough such that a reduction in the overall dosage amount given to a patient at one time can be realized. This finding should enhance patient compliance and overall satisfaction of administration.

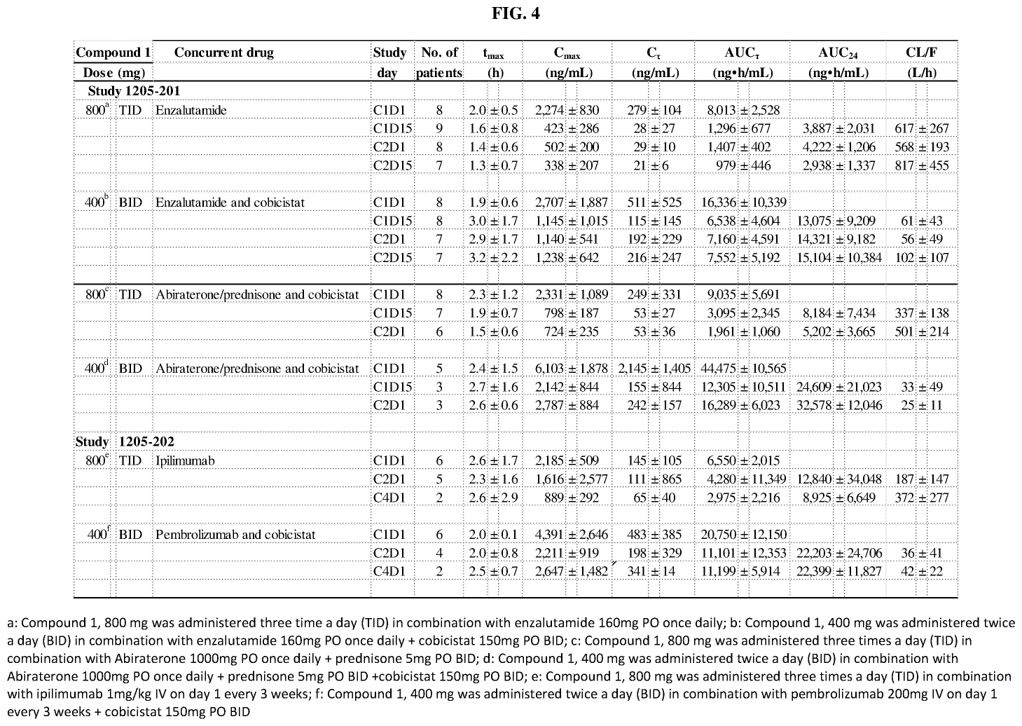

Further, these results extend to the use of cobicistat in combination with Compound 1 and other agents such as the antiandrogens en/alutarnide and abiraterone/prednisone and the immunomodulators ipilimumab and pembrolizumab. See FIG. 4.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A pharmaceutical composition comprising an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof.

2-7. (canceled)

8. A method of treating cancer in a subject in need thereof comprising the step of administering to the subject an effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or a pharmaceutically acceptable salt thereof and an effective amount of cobicistat, or a pharmaceutically acceptable salt thereof.

9. The method of claim 8, wherein the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL).

10. The method of claim 8, wherein the cancer is selected from Hodgkin's lymphoma, non-Hodgkin's lymphoma, chronic lymphocytic leukemia, and multiple myeloma.

11. The method of claim 8, wherein the cancer is a solid tumor or a lymphoma.

12. The method of claim 8, wherein the cancer is prostate cancer or non-Hodgkin's lymphoma.

13. The method of claim 8, wherein the effective amount of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, ranges from 100 mg to 3.5 gram.

14. (canceled)

15. The method of claim 8, wherein 1.6 grams of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered twice daily to the subject.

16. The method of claim 8, wherein 800 mg of (R)—N-((4-m ethoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered three times daily to the subject.

17. The method of claim 8, wherein 800 mg of (R)—N-((4-m ethoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered twice daily to the subject.

18. The method of claim 8, wherein 600 mg of (R)—N-((4-m ethoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered twice daily to the subject.

19. The method of claim 8, wherein 400 mg of (R)—N-((4-m ethoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered twice daily to the subject.

20. The method of claim 8, wherein the cancer is a lymphoma and from 100 mg to 3.5 grams of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered to the subject.

21-24. (canceled)

25. The method of claim 8, wherein the cancer is a lymphoma and 400 mg of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered twice daily to the subject.

26. The method of claim 8, wherein the cancer is a solid tumor and from 100 mg to 3.5 grams of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered to the subject.

27-30. (canceled)

31. The method of claim 8, wherein the cancer is a solid tumor and 400 mg of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof, is administered twice daily to the subject.

32. The method of claim 8, wherein the effective amount of cobicistat, or the pharmaceutically acceptable salt thereof, is from 100 mg to 400 mg.

33. (canceled)

34. The method of claim 8, wherein cobicistat, or the pharmaceutically acceptable salt thereof, is administered prior to (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, or the pharmaceutically acceptable salt thereof.
